(19) United States
(12) Patent Application Publication
(10) Pub. No.: US 2014/0059710 A1
(43) Pub. Date: Feb. 27, 2014

(54) CALIBRACHOA PLANT WITH STAR PATTERN

(71) Applicant: Plant 21 LLC, Bonsall, CA (US)

(72) Inventor: Ushio SAKAZAKI, Shiga (JP)

(73) Assignee: Plant 21 LLC, Bonsall, CA (US)

(21) Appl. No.: 13/791,614

(22) Filed: Mar. 8, 2013

Related U.S. Application Data
(63) Continuation-in-part of application No. 13/507,518, filed on Jul. 5, 2012.

(30) Foreign Application Priority Data
Mar. 9, 2012 (CA) 12-7544

(51) Int. Cl. C12N 15/82; A01H 5/02

(52) U.S. Cl. CPC C12N 15/827; A01H 5/02
USPC 800/260; 800/317; 435/430

(57) ABSTRACT

The present disclosure embraces Calibrachoa plants displaying at least one inflorescence having dark yellow colored flowers with a white-colored star pattern.

### CROSS REFERENCE TO RELATED APPLICATIONS

The present application claims priority to Canadian Plant Breeders' Rights application no. 12-______, filed Mar. 9, 2012 and U.S. Plant patent application Ser. No. ______, filed Jul. 5, 2011. Each prior application in its entirety is incorporated herein by reference.

### TECHNICAL FIELD

The present disclosure relates generally to the field of ornamental Calibrachoa plants, and provides new, distinct, and stable cultivars of Calibrachoa plants having unique inflorescence with a star pattern. Such Calibrachoa plant has not been observed in any wild species of Calibrachoa, but was discovered through Applicants' breeding process.

### INTRODUCTION

The present disclosure relates generally to the field of ornamental Calibrachoa plants. The genus Calibrachoa belongs to the Solanaceae (nightshade) family, closely related to Petunias. Many published plant taxonomies do not recognize Calibrachoa as a separate genus, and instead place it under the genus Petunia. Calibrachoa species can generally be found in South America, in southern Brazil, Peru, and Chile, in much the same regions as Petunias are found. Depending on frost conditions, Calibrachoa are considered perennials in areas that experience light frost, and annuals in other areas. While Calibrachoa produce seed, the resultant progeny generally display a range of characteristics, and so the plants are vegetatively propagated to ensure reproductions that are true-to-type of the originally selected plant.

### SUMMARY

In one aspect there is provided a Calibrachoa plant comprising at least one inflorescence with a star-shaped pattern along the center of the fused petal margins, wherein said pattern extends from the center of the inflorescence and does not fade during the life of the inflorescence. In one embodiment, the star-shaped pattern is white. In another embodiment, the star-shaped pattern extends from the center of the inflorescence to the edge of the petals. In another embodiment, the petal color is yellow.

In another aspect, there is provided a method for producing a Calibrachoa plant comprising at least one inflorescence with a star-shaped pattern along the center of the fused petal margins, comprising (a) crossing, as the male or female parent, a first Calibrachoa plant comprising at least one inflorescence with said star-shaped pattern, with a second Calibrachoa plant that does not comprise an inflorescence with said star-shaped pattern, and (b) selecting progeny that have said star-shaped pattern

wherein said star-shaped pattern extends from the center of the inflorescence towards the edge of the petals and does not fade during the life of the inflorescence.

In one embodiment, the star-shaped pattern extends from the center of the inflorescence to the edge of the petals. In another embodiment, provided is a Calibrachoa plant produced by said method, wherein tissue from said plant can be asexually propagated to produce a Calibrachoa plant comprising inflorescence with said star-shaped pattern.

In another aspect, provided is a method for asexually reproducing a Calibrachoa plant having at least one inflorescence with a star-shaped pattern along the center of the fused petal margins, comprising (a) obtaining a tissue cutting from said plant, (b) culturing said tissue cutting under conditions sufficient to produce a plantlet with roots and shoots; and (c) growing said plantlet to produce a plant, wherein said pattern extends from the center of the inflorescence towards the edge of the petals and does not fade during the life of the inflorescence. In one embodiment, said star-shaped pattern extends from the center of the inflorescence to the edge of the petals.

### BRIEF DESCRIPTION OF THE PHOTOGRAPHS

This patent or application file contains at least one drawing executed in color. Copies of this patent or patent application publication with color drawing(s) will be provided by the Office upon request and payment of necessary fee.

FIG. 1 shows a close-up view of typical inflorescences of ‘Lemon Slice.’

FIG. 2 shows a front view of a typical flowering plant of ‘Lemon Slice.’

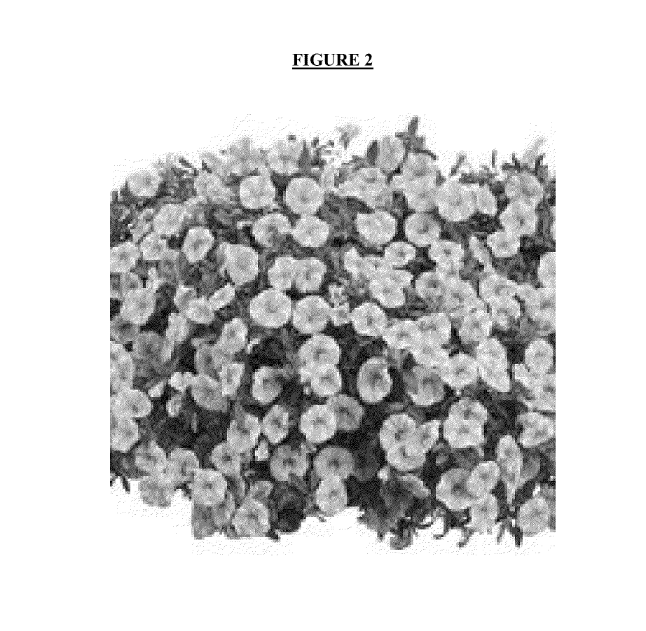

FIG. 3 shows a close-up a single inflorescence of ‘Lemon Slice.’

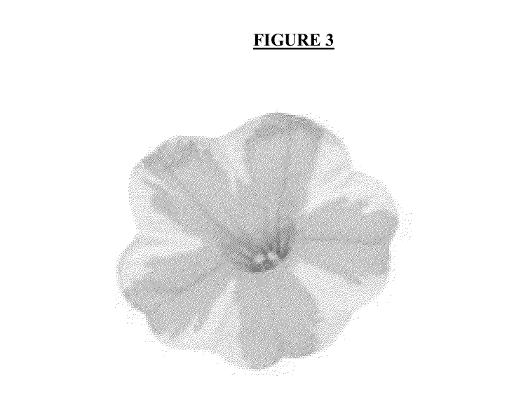

### DETAILED DESCRIPTION

The present inventors discovered a new Calibrachoa plant comprising at least one inflorescence with a star-shaped pattern along the center of the fused petal margins, wherein said pattern extends from the center of the inflorescence towards the edge of the petals and does not fade during the life of the inflorescence. The star-shaped pattern manifests itself in a white color, such that the pattern is not the same color as petal color and thereby contrasts intensely against petal color.

The petal colors of Calibrachoa are determined through the interaction of two layers of color pigment, carotenoid and anthocyanin. The carotenoid color may be yellow and the anthocyanin color may be pink, purple, or blue. The present star-shaped pattern is a result of a mutation and manifests as a white star against a yellow petal background. Regardless of the genetics, the star-shaped pattern can be reproducibly and predictably introgressed into diverse genetic backgrounds of Calibrachoa to create new varieties.

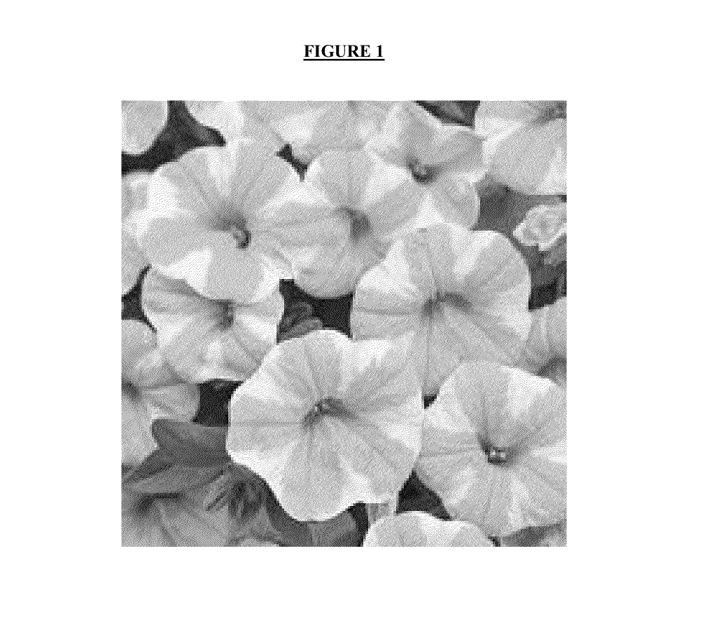

As used herein, a star-shaped pattern refers to a pigmentation pattern that appears during the bud stage and produces a clearly delineated star along each crease in the petals of each flower. The star pattern extends from the center of the inflorescence towards the edge of the petals. In some embodiments, the pattern extends all of the way to the edge of the petals. In other embodiments, the pattern may extend some distance towards the edge of the petals, but not entirely to the edge. For example, and non-limiting, a star-shaped pattern may extend less than halfway to the petals edge, more than halfway to the petals edge, or any distance between the center of the inflorescence and the edge of the petals. An exemplary star-shaped pattern can be seen in FIG. 1.

As discussed above, the star-shape can manifest as a white star. Notably, the star pattern is not the same color as petal color. For example, and in one embodiment, the star pattern is white, and the petals are yellow.

“Plant” includes plant cells, plant protoplasts, plant cells of tissue culture from which Calibrachoa plants can be regenerated from plant calli, plant clumps and plant cells that are intact in plants or parts of plants such as pollen, flowers, pistils, anthers, seeds, leaves, stems, and the like.

### A. Identification of Calibrachoa Plant ‘USCAL5302M’ or ‘Lemon Slice’

The present disclosure embraces a new and distinct cultivar of Calibrachoa plant, botanically known as Calibrachoa sp. and hereinafter referred to by the name ‘USCAL5302M’ (‘Lemon Slice’).

The new Calibrachoa plant is a naturally-occurring branch mutation of a Calibrachoa sp. ‘USCAL53002’, disclosed in U.S. Plant Pat. No. 21,660. The new Calibrachoa plant was discovered and selected by the Inventor on a single flowering plant within a population of plants of ‘USCAL53002’ in a controlled greenhouse environment in Gensingen, Germany on Jun. 8, 2010. Asexual reproduction of the new Calibrachoa plant by terminal cuttings in a controlled environment in Gensingen, Germany since Jun. 8, 2010 has shown that the unique features of this new Calibrachoa plant are stable and reproduced true to type in successive generations.

### B. Stable and Predictable Introression of the Star Pattern

The instant star pattern can be stably and predictably introgressed into diverse Calibrachoa genetic backgrounds. The instant Calibrachoa can be used as a male or female parent in crosses for introducing the trait into new Calibrachoa plants, thereby creating diverse Calibrachoa genetic backgrounds.

Additionally, and as known in the art, Calibrachoa plants can be reproduced asexually by vegetative propagation or other clonal method known in the art. For example, and in no way limiting, a Calibrachoa plant having at least one inflorescence with the instant dark yellow flower color and white star pattern, can be reproduced by (a) obtaining a tissue cutting from said plant, (b) culturing said tissue cutting under conditions sufficient to produce a plantlet with roots and shoots; and (c) growing said plantlet to produce a plant.

The following Examples are illustrative and do not limit the disclosure.

### EXAMPLE 1

### Botanical Description of ‘USCAL5302M’ (‘Lemon Slice’)

The aforementioned photographs and following observations, measurements, and values describe plants grown during the spring in 11.4-cm containers in a polyethylene-covered greenhouse and finished in an outdoor nursery in Bonsall, Calif.

During the production of the plants, day temperatures averaged 24° C. and night temperatures averaged 18° C. Plants were pinched one time and were seven weeks old when the photographs and description were taken. In the following description, color references are made to The Royal Horticultural Society Colour Chart, 2007 Edition, except where general terms of ordinary dictionary significance are used.

### Botanical Classification:

Calibrachoa sp. ‘USCAL5302M’

### Parentage:

Naturally occurring branch mutation of a Calibrachoa sp. ‘USCAL53002,’ disclosed in U.S. Plant Pat. No. 21,660.

### Propagation:

Type: By terminal cuttings.
Time to initiate roots, summer: About four days at temperatures ranging from 17° C. to 29° C.
Time to initiate roots, winter: About six days at temperatures ranging from 17° C. to 21° C.
Time to produce a rooted plant, summer: About 22 days at temperatures ranging from 17° C. to 29° C.
Time to produce a rooted plant, winter: About 25 days at temperatures ranging from 17° C. to 21° C.
Root description: Medium in thickness, fibrous; white in color.
Rooting habit: Freely branching; medium density.

### Plant Description:

Plant and growth habit: Upright, outwardly spreading to trailing and mounding plant habit; freely branching habit with about seven primary lateral branches each with multiple secondary lateral branches; pinching enhances branching; moderately vigorous, dense and bushy growth habit.
Plant height: About 12.5 cm.
Plant diameter: About 26 cm by 30 cm.

### Lateral Branch Description:

Length: About 17.8 cm.
Diameter: About 2 mm.
Internode length, About 1.7 cm.
Strength: Strong.
Aspect: Initially upright to outwardly spreading to trailing.
Texture: Pubescent.
Color: Close to 146D tinted with close to 187B.

### Foliage Description:

Arrangement: Before flowering, alternate; after flowering, mostly alternate to nearly opposite; leaves simple.
Length: About 4.7 cm.
Width: About 2 cm.
Shape: Elliptical.
Apex: Broadly acute.
Base: Attenuate.
Margin: Entire.
Texture, upper and lower surfaces: Pubescent.
Venation pattern: Pinnate; arcuate.

Color:
Developing leaves, upper surfaces: Close to 137 A.
Developing leaves, lower surfaces: Close to 146A.
Fully expanded leaves, upper surface: Close to N137A; venation, close to 138A.
Fully expanded leaves, lower surface: Close to 147B; venation, close to 138A.

Petiole:
Length: About 3 mm.
Diameter: About 2 mm.
Texture, upper and lower surfaces: Pubescent.
Color, upper and lower surface: Close to 146D.

### Flower Description:

Flower arrangement and habit: Single salverform flowers arising from leaf axils; freely flowering habit with usually about 42 open flowers and flower buds per lateral branch; flowers face upright to mostly outwardly.
Fragrance: None detected.
Natural flowering season: Early flowering habit, plants of the new Calibrachoa begin flowering about six weeks after planting; plants flower continuously from spring though the autumn in Southern California.
Flower longevity: Individual flowers last about four to five days on the plant; flowers persistent.
Flower diameter: About 3.7 cm.
Flower length (height): About 3.2 cm.
Flower throat diameter: About 4 mm by 8 mm.
Flower tube length: About 2 cm.
Flower tube diameter, base: About 2.5 mm.

Flower bud:
Shape: Elongated oblong.
Length: About 2.9 cm.
Diameter: About 6 mm.
Color: Close to 151D.

Corolla:
Arrangement: Five petals fused at the base and opening into a flared trumpet.
Petal lobe length (from throat): About 1.4 cm.
Petal lobe width: About 1.9 cm.
Petal shape: Roughly obovate to spatulate.
Petal apex: Rounded.
Petal margin: Entire.
Petal texture, upper surface: Smooth, glabrous
Petal texture, lower surface: Smooth, mostly glabrous with a few hairs along the veins.
Throat texture: Smooth, glabrous.
Tube texture: Pubescent.

Color:
Petal lobe, when opening, upper surface: Close to 7A; star pattern, close to NN155D
Petal lobe, when opening, lower surface: Close to 4C
Petal lobe, fully opened, upper surface: Close to 14B; star pattern, close to NN155D; venation, close to 145B; color does not fade with development.
Petal lobe, fully opened, lower surface: Close to 8C; star pattern, close to NN155D; venation, close to 145A.
Throat: Close to 7A; venation, 5 close to 153A.
Tube: Close to 4B; venation, close to 145A.

Calyx:
Arrangement: One star-shaped calyx tube with five sepals fused towards the base and arranged in a single whorl.
Sepal length: About 1.4 cm.
Sepal width: About 3 mm.
Sepal shape: Lanceolate.
Sepal apex: Acute.
Sepal margin: Entire.
Sepal texture, upper and lower surfaces: Pubescent.
Color, upper and lower surfaces: Close to 138A to 138B.

Peduncles:
Length: About 2.2 cm.
Diameter: About 1 mm.
Angle: About 45° from stem axis.
Strength: Strong.
Texture: Pubescent.
Color: Close to 146C.

Reproductive Organs:

Stamens:
Quantity: About five per flower.
Filament length: About 1 cm.
Filament color: Close to 145C.
Anther shape: Oval.
Anther length: About 1 mm.
Anther color: Close to 12A.
Pollen amount: Scarce.
Pollen color: Close to 12C.

Pistils:
Quantity: One per flower.
Pistil length: About 1 cm.
Style length: About 8 mm.
Style color: Close to 145C.
Stigma shape: Round.
Stigma color: Close to 146C.
Ovary color: Close to 146D.

Seeds and Fruits:
Seed and fruit development have not been observed on plants of the new Calibrachoa.

### Garden Performance:

Plants of the new Calibrachoa have been observed to have good garden performance and tolerate wind, rain and temperatures ranging from about 1° C. to about 40° C.

### Pathogen/Pest Resistance:

Plants of the new Calibrachoa have not been observed to be resistant to pathogens and pests common to Calibrachoa.

### EXAMPLE 2

### Comparison of ‘Lemon Slice’ with ‘USCAL53002’

The closest known variety to ‘Lemon Slice’ is parent plant ‘USCAL53002.’ ‘Lemon Slice’ differs from parent plant ‘USCAL53002’ primarily in flower color and pattern. Parent ‘USCAL53002’ has solid dark yellow-colored flowers and does not have a star-shaped pattern. Thus, in comparison, ‘Lemon Slice’ has a dark yellow flowers with a white star-shaped pattern.

### EXAMPLE 3

### Comparison of ‘Lemon Slice’ with ‘USCALI402-1’

The instant ‘Lemon Slice’ Calibrachoa can be compared to plants of Calibrachoa ‘USCALI402-1’, as disclosed in U.S. Plant Pat. No. 10 19,480. In side-by-side comparisons conducted in Bonsall, Calif., plants of the new Calibrachoa differed primarily from plants of ‘USCALI402-1’ in flower color as plants of ‘USCALI402-1’ had pale yellow-colored flowers.

### EXAMPLE 4

### Stable and Predictable Introression of the Star Pattern

The instant star pattern can be stably and predictably introgressed into diverse Calibrachoa genetic backgrounds. The instant Calibrachoa can be used as a male or female parent in crosses for introducing the trait into new Calibrachoa plants, thereby creating diverse Calibrachoa genetic backgrounds.

Additionally, and as known in the art, Calibrachoa plants can be reproduced asexually by vegetative propagation or other clonal method known in the art. For example, and in no way limiting, a Calibrachoa plant having at least one inflorescence with the instant dark yellow flower color and white star pattern, can be reproduced by (a) obtaining a tissue cutting from said plant, (b) culturing said tissue cutting under conditions sufficient to produce a plantlet with roots and shoots; and (c) growing said plantlet to produce a plant.

What is claimed is:

1. A Calibrachoa plant comprising at least one inflorescence with a star-shaped pattern along the center of the fused petal margins, wherein said pattern extends from the center of the inflorescence and does not fade during the life of the inflorescence.

2. The Calibrachoa plant of claim 1, wherein said star-shaped pattern is white.

3. The Calibrachoa plant of claim 1, wherein said star-shaped pattern extends from the center of the inflorescence to the edge of the petals.

4. The Calibrachoa plant of claim 1, wherein said petal color is yellow.

5. A method for producing a Calibrachoa plant comprising at least one inflorescence with a star-shaped pattern along the center of the fused petal margins, comprising
(a) crossing, as the male or female parent, a first Calibrachoa plant comprising at least one inflorescence with said star-shaped pattern, with a second Calibrachoa plant that does not comprise an inflorescence with said star-shaped pattern, and
(b) selecting progeny that have said star-shaped pattern
wherein said star-shaped pattern extends from the center of the inflorescence towards the edge of the petals and does not fade during the life of the inflorescence.

6. The method of claim 5, wherein said star-shaped pattern extends from the center of the inflorescence to the edge of the petals.

7. A Calibrachoa plant produced by the method of claim 5, wherein tissue from said plant can be asexually propagated to produce a Calibrachoa plant comprising inflorescence with said star-shaped pattern.

8. A method for asexually reproducing a Calibrachoa plant having at least one
inflorescence with a star-shaped pattern along the center of the fused petal margins, comprising
(a) obtaining a tissue cutting from said plant,
(b) culturing said tissue cutting under conditions sufficient to produce a plantlet with roots and shoots; and
(c) growing said plantlet to produce a plant,
wherein said pattern extends from the center of the inflorescence towards the edge of the petals and does not fade during the life of the inflorescence.

9. The method of claim 8, wherein said star-shaped pattern extends from the center of the inflorescence to the edge of the petals.
